(19) United States
(12) Patent Application Publication
(10) Pub. No.: US 2009/0299170 A1
(43) Pub. Date: Dec. 3, 2009

(54) Magnetic resonance scanner with PET unit

(75) Inventors: Matthias Gebhardt, Erlangen (DE); Wolfgang Renz, Erlangen (DE); Sebastian Schmidt, Weisendorf (DE)

Correspondence Address: HARNESS, DICKEY & PIERCE, P.L.C., P.O. BOX 8910, RESTON, VA, 20195, US

(73) Assignee: Siemens Aktiengesellschaft

(21) Appl. No.: 12/453,972

(22) Filed: May 28, 2009

(30) Foreign Application Priority Data
May 29, 2008 (DE) 10 2008 025 677.3

(51) Int. Cl. A61B 5/055

(52) U.S. Cl. 600/411

(57) ABSTRACT

A magnetic resonance scanner including a PET unit includes a magnet system and a gradient system having a patient bore. In at least one embodiment, the magnet system and the gradient system are each split by an azimuthal gap and the PET unit is disposed within the gap.

### PRIORITY STATEMENT

The present application hereby claims priority under 35 U.S.C. §119 on German patent application number DE 10 2008 025 677.3 filed May 29, 2008, the entire contents of which is hereby incorporated herein by reference.

### FIELD

At least one embodiment of the present invention generally relates to a magnetic resonance scanner having a PET unit, comprising a magnet system and a gradient system having a patient bore, wherein the magnet system and the gradient system are each split by an azimuthal gap.

### BACKGROUND

In addition to magnetic resonance tomography (MR), in recent years positron emission tomography (PET) has also been used ever more widely in medical diagnosis. While MR is an imaging method for representing structures and slices inside the body, PET allows in vivo visualization and quantification of metabolic activities. However, although PET is highly sensitive, it only provides low spatial resolution. The latter cannot be improved at will because of several effects.

On the one hand, the method measures the position of positron annihilation. However, the positron covers a finite distance from creation to annihilation. The average free path length depends on the radionuclide and is in the millimeter range. Moreover, the photons are not emitted in a precisely colinear manner, but with a minimal deviation from the 180° angle, what is referred to as the colinearity error. Lastly, the size of the scintillation crystals cannot be reduced at will, as this reduces the sensitivity and also increases the manufacturing costs.

PET uses the particular properties of positron emitters and positron annihilation in order to quantitatively determine the function of organs or cell areas. With this technique, appropriate radiopharmaceuticals marked with radionuclides are administered to the patient prior to the examination. As they decay, the radionuclides emit positrons which after a short distance interact with an electron, causing what is termed annihilation to occur. This results in two gamma quanta which fly apart in opposite directions (offset by 180°). The gamma quanta are detected by two opposing PET detector modules within a particular time window (coincidence measurement), by means of which the annihilation site is localized to a position on the line connecting said two detector modules.

In the case of PET, the detector module must generally cover the greater part of the gantry arc length for the purpose of detection. It is subdivided into detector elements having a side length of a few millimeters. On detecting a gamma quantum, each detector element generates an event record that specifies the time and the detection location, i.e. the corresponding detector element. These items of information are transmitted to a fast logic unit and compared. If two events coincide within a maximum time period, it is assumed that there is a gamma decay process on the connecting line between the two associated detector elements. The PET image is reconstructed using a tomography algorithm, i.e. so called back-projection.

In order to compensate for PET's lack of spatial resolution, combined PET/CT scanners can be used, a PET scanner and a CT scanner being disposed back-to-back such that the patient can be transferred seamlessly from one scanner to the other within one examination. The two measurements can then take place in immediate succession. However, in such systems it is not possible for PET and CT data to be measured simultaneously.

It is advantageous to combine a PET scanner with an MR scanner, as MR provides a higher soft tissue contrast compared to CT. Combined MR/PET systems are already known in which the PET detectors are disposed, together with the gradient system and excitation coil, inside an aperture defined by the MR magnets. In this configuration, they are positioned next to the excitation coil so that the scanning volumes of the MR and PET system do not coincide, but are offset in the Z-direction. Analogously to the PET/CT system, the PET and MR data cannot therefore be measured simultaneously here.

In this context, it would be particularly preferable for the PET scanner to be disposed inside the MR scanner and the two scanning volumes to be superimposed. In this case both morphological MR data and PET data can be determined within one measuring pass. In addition to the time-saving effect, motion correction of the PET data can be performed, among other things, on the basis of the measured MR data, as disclosed in DE 10 2005 023 907 A1, the entire contents of which is hereby incorporated herein by reference. The two image datasets can also be simply displayed in a superimposed manner so as to facilitate the physician's diagnostic assessment.

To integrate the PET and MR scanners, it is necessary to dispose the PET detectors inside the MR scanner so that the imaging volumes are ideally isocentric. For example, the PET detectors can be disposed on a supporting structure (supporting tube, gantry) inside the MR scanner. For example, 60 detectors can be disposed in an annular arrangement on the supporting tube. A cooling connection and electrical leads are required for each of the detectors, which can also be combined to form detector blocks. The cooling connection and electrical leads must likewise be disposed in the MR scanner. Also required are a number of signal processing units which are likewise disposed in the MR scanner. These are connected to the detectors via the electrical leads and are used for signal processing.

WO 2006/071922 A2 discloses a combined MR/PET scanner in which a detector array is disposed inside an MR magnet. The isocentric arrangement of the scanning volumes enables MR and PET data to be acquired simultaneously. In this case, however, the available patient bore is reduced by the incorporation of the detectors. Particularly if a whole body scan is to be performable using the MR/PET scanner, a maximally large patient bore is required. The PET detectors must additionally have a minimum depth in order to capture the gamma quanta with sufficiently high probability. Consequently, the thickness of the detectors cannot be reduced at will.

MR systems are also known in which an intervention, e.g. by a physician, is possible during a scan. For example, open gradient coils for MR systems are disclosed in U.S. Pat. No. 5,378,989 and U.S. Pat. No. 5,952,830. The gradient coils are here of two-part design, the sections being disposed separated from one another by an azimuthal gap. A patient positioned in the patient bore is therefore externally accessible, thereby allowing an intervention during or immediately prior to MR data measurement. However, the MR systems described are not designed for measuring PET data.

### SUMMARY

In at least one embodiment of the present invention provides a magnetic resonance scanner incorporating a PET unit, wherein the scanning volumes of the magnetic resonance scanner and PET unit are at least partially identical and yet a maximally large patient bore can be provided.

According to one embodiment variant of the invention, a magnetic resonance scanner incorporating a PET unit is provided, comprising a magnet system and a gradient system having a patient bore. The magnet system and the gradient system are each split by an azimuthal gap. The PET unit is disposed inside the gap. An advantage of at least one embodiment of the inventive disposition of the PET unit is that a patient bore comparable to that of an MR system without PET unit is available. As is known in the prior art, an excitation coil of the MR system can be disposed next to the PET unit as a means of avoiding space problems. In contrast to the known prior art system with adjacently disposed excitation coil and PET unit, in at least one embodiment of the present invention the scanning volumes of the MR system and PET system are disposed isocentrically, thereby enabling MR and PET data to be measured simultaneously.

In an advantageous embodiment of the invention, the magnet system has a supporting structure with magnet coils which extends across the gap and on which the PET unit is disposed. The described design of the magnet system allows the PET system's detection units to be disposed on the same supporting structure as that used for the magnet coils, thereby simultaneously achieving increased system stability and a simple design.

In another advantageous embodiment of the invention, the gradient system has a supporting structure for gradient coils which extends across the gap and on which the PET unit is disposed. Also in this variant of an embodiment of the invention, a simplified system design is achieved by the continuous supporting structure.

In another advantageous embodiment of the invention, the magnetic resonance scanner incorporates a supporting structure for an excitation coil which extends across the gap and on which the PET unit is disposed, again resulting in the advantage of simplified system design.

In an advantageous embodiment of the invention, the PET unit has a plurality of detection units which are disposed around the patient bore in an annular manner. This design of the PET unit is particularly suitable for placing in the gap between the two sections of the magnet system and of the gradient coils. The annular arrangement also offers advantages in respect of signal delay, which is particularly important in the case of time-critical PET measurements.

One embodiment of the invention is advantageous in that the detectors disposed in an annular manner have a recess through which access to the patient bore is defined. Compared to known open MR systems, this makes it unnecessary to forego the possibility of intervention on the patient during the MR or PET scan.

One embodiment of the invention is advantageous in that the PET unit incorporates a processing unit for PET data which is disposed in an annular manner around the detection units. In the gap provided there is generally much more space than inside the gradient coils or the excitation coil in known MR systems, thus making it possible for processing units for the PET data to be disposed near the PET units. This significantly reduces the overall system design complexity. In addition, the PET data can be suitably conditioned by the processing unit for transmission to a computer, so that effects of the MR system on data transmission are largely avoided.

In an advantageous embodiment of the invention, the magnet system comprises a cooling unit and two cooling tanks separated by the gap. The cooling tanks are connected by a thermal bridge such that they can be cooled by the cooling unit. This simplifies system design, as only one cooling unit needs to be provided for cooling the magnet system.

An embodiment of the invention is advantageous in that the thermal bridge is formed by a cavity filled with liquid helium. This design of the thermal bridge is particularly simple to implement.

An embodiment of the invention is advantageous in that the magnet system comprises two superconducting coil sections separated by the gap which are electrically connected by a superconducting line. This simplifies the design of the magnet system, as the corresponding coil sections act like a single coil without impairing the separation.

### BRIEF DESCRIPTION OF THE DRAWINGS

Further advantages and embodiments of the invention will emerge from the following description of example embodiments with reference to the accompanying drawings, in which:

FIG. 1 shows a known type of combined MR/PET scanner,

FIG. 2 shows a known type of split MR system, and

FIGS. 3 to 5 show different embodiment variants of the invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The example embodiments of the invention can preferably be used on a combined MR/PET scanner. The advantage of a combined scanner is that both MR and PET data can be acquired isocentrically. This enables the scanning volume to be precisely defined within the region of interest using the data of the first modality (PET) and this information to be used in the other modality (e.g. magnetic resonance). Although it is possible for the volume information of the region of interest to be transferred from an external PET to an MR scanner, this entails increased overhead in terms of the registration of the data.

In general, all the data that can be determined using magnetic resonance or other imaging methods can be ascertained from the region of interest selected on the PET dataset. For example, instead of the spectroscopy data, fMR data, diffusion maps, T1- or T2-weighted images or quantitative parameter maps can also be obtained by means of magnetic resonance scans in the region of interest. Computed tomography methods (e.g. perfusion measurement, multi-energy imaging) or X-rays can likewise be used. In each case the described method has the advantage that the region of interest can very selectively narrowed down by means of the PET dataset to a specifically present patient pathology.

In addition it is, however, also possible, by using a plurality of what are termed tracers, to represent different biological properties in the PET dataset and thus optimize still further the region of interest and the volume defined thereby or select a plurality of different scanning volumes at once which are then analyzed in subsequent scans.

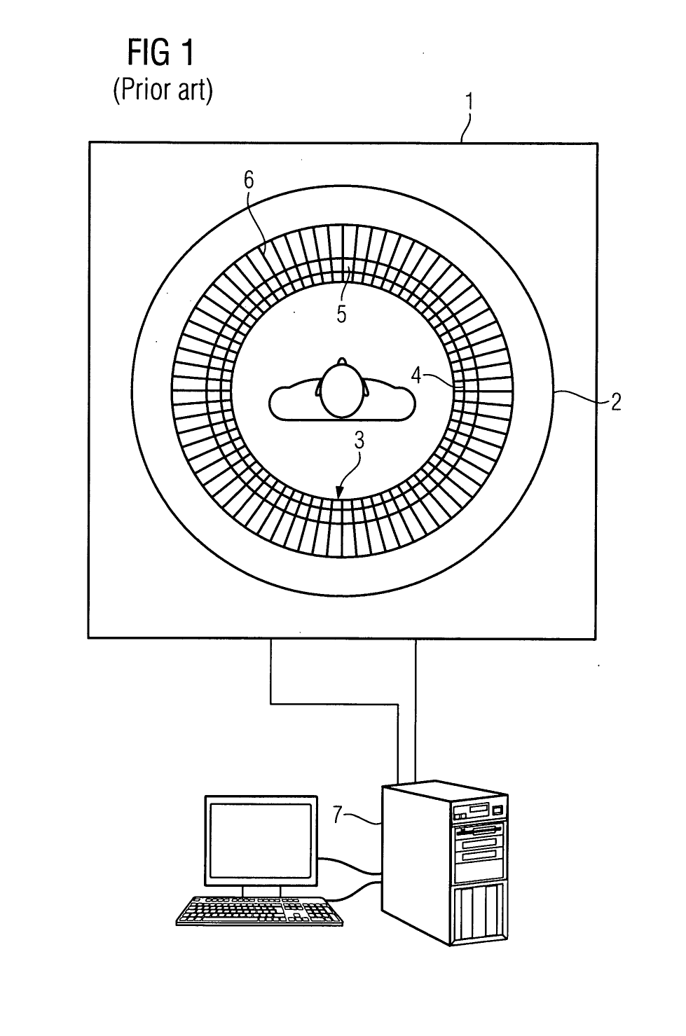

FIG. 1 shows a known device 1 for superimposed MR and PET image representation. The device 1 consists of a known MR tube 2. The MR tube 2 defines a longitudinal direction z which extends orthogonally to the drawing plane of FIG. 1.

As shown in FIG. 1, a plurality of PET detection units 3 arranged in opposing pairs about the longitudinal direction z are disposed coaxially inside the MR tube 2. The PET detection units 3 preferably consist of an APD photodiode array 5 preceded by an array of LSO crystals 4 and an electrical amplifier circuit (AMP) 6. However, the embodiments of the invention is not limited to the PET detection units 3 having the APD photodiode array 5 preceded by an array of LSO crystals 4, but other kinds of photodiodes, crystals and devices can equally be used for detection purposes.

Image processing for superimposed MR and PET image representation is performed by a computer 7.

Along its longitudinal direction z, the MR tube 2 defines a cylindrical first field of view. The plurality of PET detection units 3 define, along the longitudinal direction z, a cylindrical second field of view. According to an embodiment of the invention, the second field of view of the PET detection units 3 essentially coincides with the first field of view of the MR tube 2. This is implemented by appropriately adapting the arrangement density of the PET detection units 3 along the longitudinal direction z.

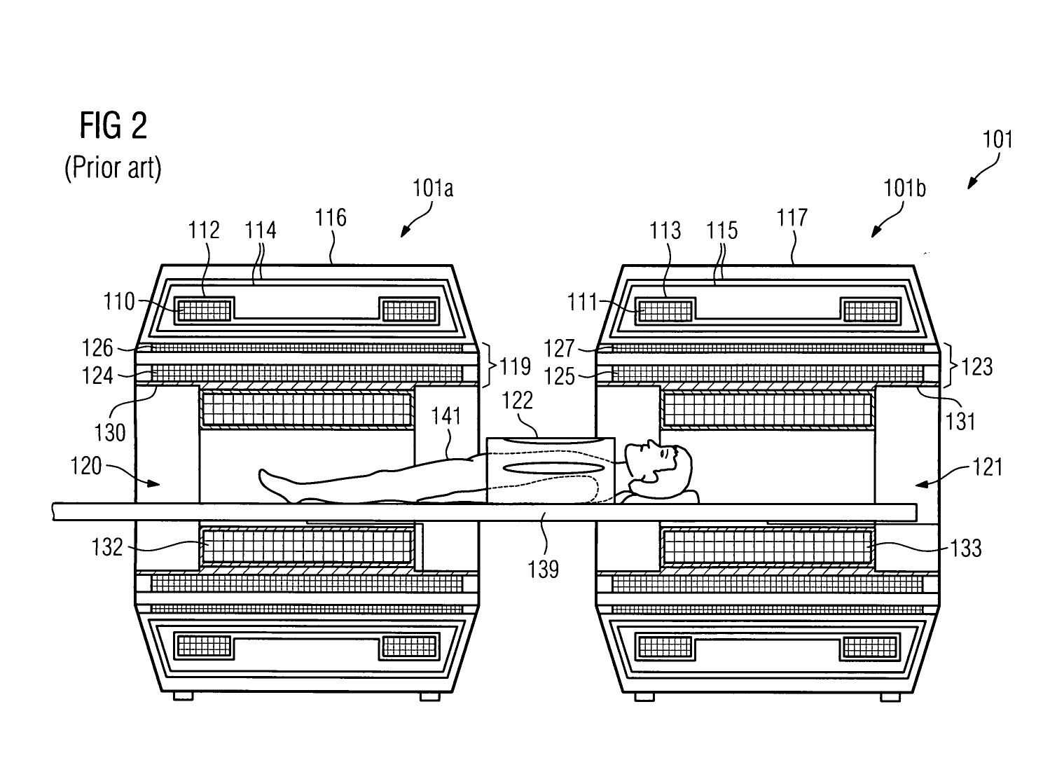

FIG. 2 shows a known split MR system 101 in a cross-sectional view. It is split into two sections 101a and 101b and consequently comprises the usual MR system components in duplicate. To provide the main magnetic field, the MR system 101 comprises two main magnets 110 and 111, which are preferably implemented as superconducting coils. In this case the main magnets 110 and 111 are disposed in helium reservoirs 112 and 113 containing liquid helium for maintaining the superconducting state. The helium reservoirs 112 and 113 are surrounded by a plurality of cold shields 114 and 115 which protect them against heat from outside, thereby preventing evaporation of the liquid helium. The entire magnet arrangement is surrounded by vacuum chambers 116 and 117. Through the vacuum chambers 116 and 117, openings 120 and 121 respectively are defined, inside which gradient coil units 119 and 123 are disposed. The latter are preferably designed as whole-body gradient coils.

In the example embodiment shown, the gradient coil units 119 and 123 each include a primary coil 124 and 125 respectively, by means of which gradient fields can be provided in the x-, y- and z-direction. Additionally provided are two shield coils 126 and 127 which are disposed inside the vacuum chambers 116 and 117. The magnetic field produced by the primary coils 124 and 125 is nullified by them inside the vacuum vessels 117 and 116 respectively, so that the main magnets 110 and 111 are unaffected. Disposed inside the primary coils 124 and 125 are RF shields 130 and 131 respectively which are transparent to the gradient fields generated (typically in the kHz range) but opaque to frequencies in the MHz range. Disposed inside the RF shields 130 and 131 are two excitation coils 132 and 133 respectively, by means of which magnetic resonance can be excited and measured inside a patient. In the remaining part of the openings 120 and 121, a continuous patient positioning table 139 is provided on which a patient 141 can be moved through the system. It is optionally possible to provide the patient 141 with a local coil 122.

Because of its split design, the MR system 101 described here enables an intervention to be performed on the patient 141 during scans or between scans. Thus, for example, contrast agents can be administered or radiation treatment given. It is likewise possible to carry out catheter examinations, it then being possible for the catheters to be precisely positioned using the MR system 101.

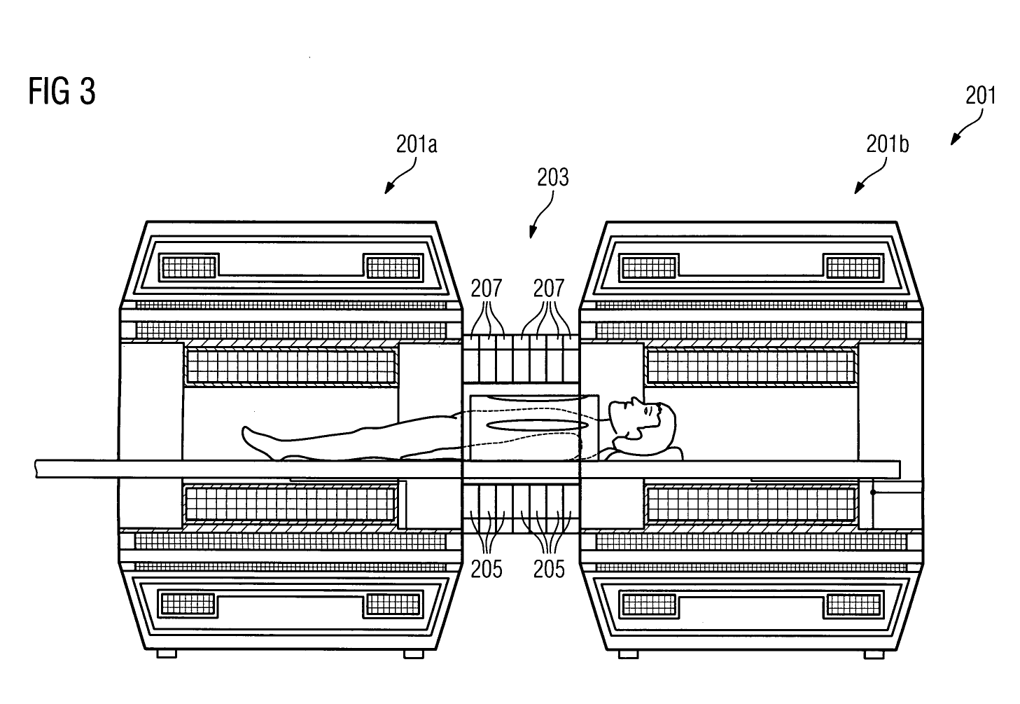

FIG. 3 shows an MR/PET system 201 as an example embodiment of the invention. It includes a split MR system of basically similar design to that of the MR system 101 illustrated in FIG. 2. However, a PET gantry 203 is disposed between the two sections 201a and 210b. The scanning volume of the PET gantry 203 coincides with the common scanning volume of the two sections 201a and 201b. The design of the PET gantry 203 is comparable to the design shown in FIG. 1. It consists of a plurality of detection units 205 and their associated processing units 207. With the system shown in FIG. 3, MR and PET data can be measured simultaneously without having to accept limitations in respect of the size of the available patient bore.

In addition, in the example described here it is possible for the PET gantry 203 to be easily accessed from outside. As frequent maintenance is necessary here, ease of access to the detection units 205 and the processing units 207 compared to a fully integrated PET system (as in FIG. 1) significantly reduces maintenance costs and complexity. Moreover, individual detection units 205 can be replaced in a simple manner. With a corresponding design, it is also easy to completely or partially remove the PET system so as to allow an intervention as in the MR system in FIG. 2. As an alternative to the PET gantry 203, another image generating system can preferably be inserted between the sections 201a and 201b of the MR/PET system. Fluorescence imaging, for example, can then be used simultaneously with MR. The PET gantry 203 would merely have to be replaced by corresponding optical detectors, thereby considerably increasing the flexibility of the system.

Also, due to the open access to the PET system, more space is available for routing transmission lines and cooling lines, as these no longer have to be incorporated in the patient bore.

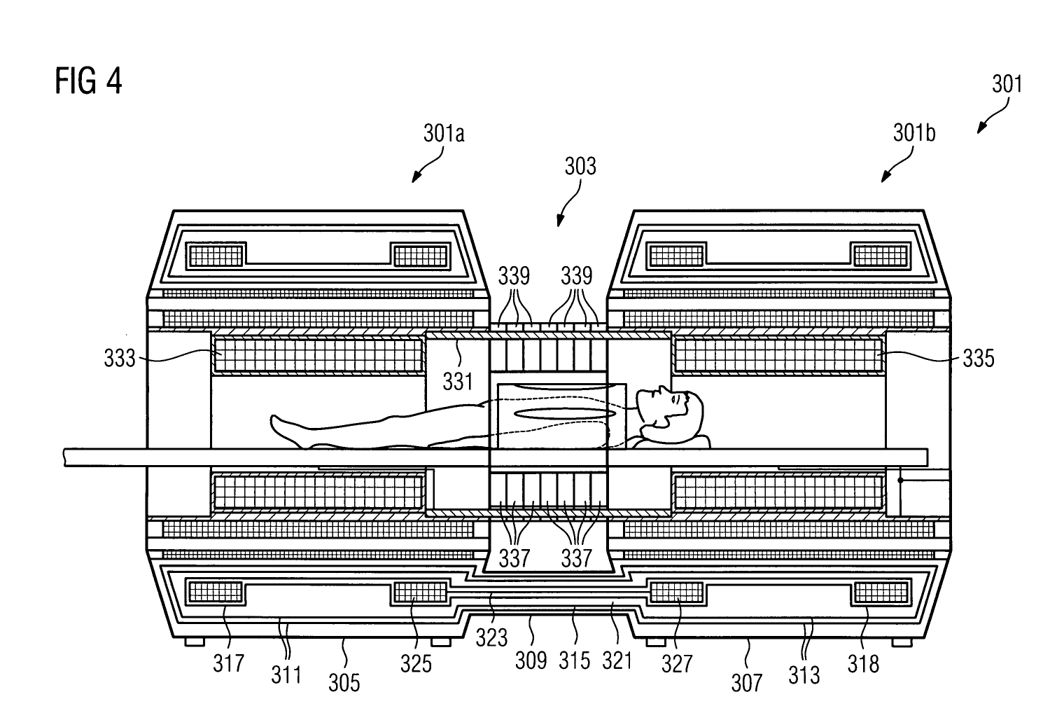

FIG. 4 shows an alternative embodiment variant of the invention. The MR/PET system 301 illustrated is largely similar to the exemplary embodiment shown in FIG. 3. In particular, the MR part of the MR/PET system 301 consists of two sections 301a and 301b that are set up spaced apart from each other. A PET gantry 303 is disposed between the sections 301a and 301b. Simultaneous measurement of MR and PET data is also possible with this embodiment variant of the invention, as the two system components have superimposed scanning volumes. The difference compared to the embodiment variant shown in FIG. 3 is that the physical separation of the two sections 301a and 301b is not complete. In fact, two vacuum chambers 305 and 307 of the two sections 301a and 301b are connected via a vacuum duct 309. The same applies to the cold shields 311 and 313 which are likewise connected by shield bridges 315 in each case. Helium vessels 317 and 318 of the sections 301a and 301b are connected via a helium duct 321. Inside the helium duct 321, a superconducting connecting line 323 is provided by means of which the superconducting coils of the two main magnets 325 and 327 are interconnected. The advantage of this embodiment variant is that the two main magnets 325 and 327 can be controlled as one magnet, analogously to non-split MR systems. Connecting the two helium vessels 317 and 318 makes it possible for the two main magnets 325 and 327 to be cooled by a single cooling system (not shown here).

Independently of the coupling of the two magnetic field systems and of the corresponding shields and vacuum chambers shown, the PET gantry 303 is disposed on a supporting structure 331 which also supports the excitation coils 333 and 335 of the two sections 301a and 301b. This simplifies the design of the system. The arrangement of the PET gantry 303 on the supporting structure 331 is selected such that the detection units 337 are retained inside the supporting structure 331 while the processing units 339 are disposed outside the supporting structure. The detection units 337 and the processing units 339 are consequently disposed on opposite sides of the supporting structure 331, thereby facilitating access to the processing units 339 for maintenance purposes or to connect leads, for example.

Alternatively, depending on the system design, the components of the PET gantry 303 can also be disposed completely on the inner or outer side of the supporting structure 331. In the latter case, the supporting structure 331 must be made of a material which attenuates gamma quanta as little as possible. Options here include, for example, carbon or glass-reinforced plastic (GRP). It is likewise possible to dispose the components of the PET gantry 303 on a common supporting structure with the components of the gradient system or magnet coils.

As a variation of the embodiments of the invention shown in FIGS. 3 and 4, it is possible to move the components of the sections 301a and 301b of the MR system closer to the PET gantry 303 so as to optimize imaging in the field of view of the PET gantry 303. The windings of the magnets can be optimized accordingly.

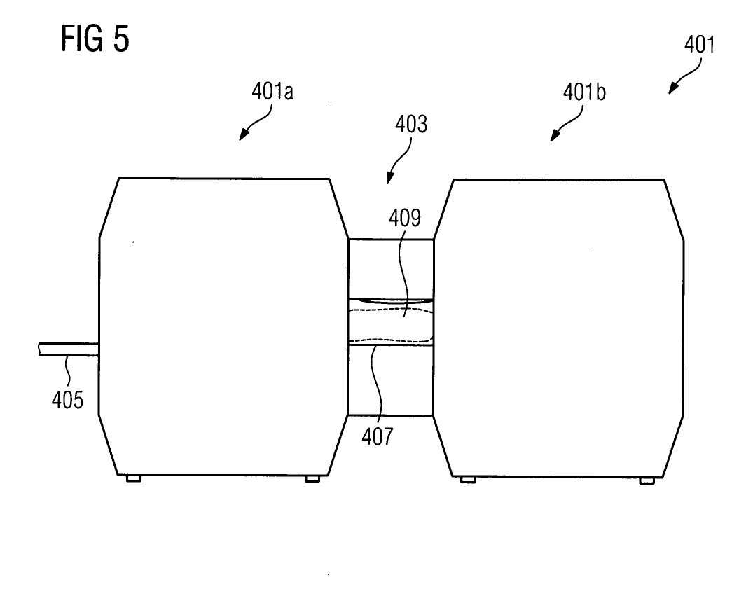

FIG. 5 shows another alternative embodiment variant of the invention. The MR/PET system 401 illustrated is again largely similar to the embodiment variant shown in FIG. 3. In contrast to the illustrations in FIGS. 3 and 4, in FIG. 5 a side view rather than a cross section is shown for the sake of clarity of representation of this embodiment variant. However, the internal design of the MR/PET system 401 is largely identical to the exemplary embodiments shown in FIGS. 3 or 4. However, the connection of the two magnet systems as in FIG. 4 has been omitted. The MR/PET system 401 shown consists of two sections 401a and 401b which are arranged spaced apart from each other. A patient positioning table 405 is disposed inside the sections 401a and 401b.

A PET gantry 403 is disposed between the two sections 401a and 401b. The PET gantry 403 has a lateral recess 407 affording access to a patient 409. Consequently, with this embodiment variant of the invention, an intervention on the patient is possible during scanning or between individual scans, e.g. to administer a contrast agent or give radiation treatment to the patient. A recess 407 of this kind is also possible in the examples in FIGS. 3 and 4. It is likewise possible to create the recess 407 as required by removing one or more detection units of the PET unit. These can be reinstalled if the recess 407 is not required.

In the embodiment variants of the invention shown, a split gradient system is provided in each case. This is preferably designed to minimize the Lorentz forces acting on the individual system components as a result of the rapid field changes.

The processing units are preferably disposed annularly around the detection units, as also shown in FIGS. 3 and 4. The advantage of this is that the line lengths and therefore the signal delays to an evaluation computer are of the same length, which is particularly important in the case of time-critical PET measurements (e.g. time-of-flight measurements).

It is likewise possible to dispose the two sections of the MR system and the PET system in a single vacuum chamber. The advantage of this is that the scanner only needs one vacuum chamber and the manufacturing costs are reduced. It is advantageous here if the material selected for the casing of the vacuum vessel attenuates gamma quanta as little as possible. Possible materials here are likewise GRP or carbon.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A magnetic resonance scanner including a PET unit, comprising:
a magnet system; and
a gradient system including a patient bore, the magnet system and the gradient system each being split by an azimuthal gap, the PET unit being disposed within the gap.

2. The magnetic resonance scanner as claimed in claim 1, wherein the magnet system includes a supporting structure, for magnet coils, which extends across the azimuthal gap and on which the PET unit is disposed.

3. The magnetic resonance scanner as claimed in claim 1, wherein the gradient system includes a supporting structure, for gradient coils, which extends across the azimuthal gap and on which the PET unit is disposed.

4. The magnetic resonance scanner as claimed in claim 1, comprising:
a supporting structure, for excitation coils, which extends across the azimuthal gap and on which the PET unit is disposed.

5. The magnetic resonance scanner as claimed in claim 1, wherein the PET unit includes a plurality of detection units, disposed annularly around the patient bore.

6. The magnetic resonance scanner as claimed in claim 5, wherein the annularly disposed detection units include a recess through which access to the patient bore is defined.

7. The magnetic resonance scanner as claimed in claim 5, wherein the PET unit includes at least one processing unit for PET data, disposed annularly around the detection units.

8. The magnetic resonance scanner as claimed in claim 5, wherein the detection units are disposed in a detachable manner.

9. The magnetic resonance scanner as claimed in claim 1, wherein the PET unit comprises a coolant feed.

10. The magnetic resonance scanner as claimed in claim 1, wherein the magnet system comprises a cooling unit and two cooling tanks separated by the gap, the cooling tanks being connected by a thermal bridge such that the cooling tanks can be cooled by the cooling unit.

11. The magnetic resonance scanner as claimed in claim 10, wherein the thermal bridge is formed by a cavity filled with liquid helium.

12. The magnetic resonance scanner as claimed in claim 11, wherein the magnet system comprises two superconducting coil sections separated by the gap which are electrically connected by a superconducting line.

13. The magnetic resonance scanner as claimed in claim 12, wherein the line runs inside the cavity.

14. The magnetic resonance scanner as claimed in claim 1, wherein the gradient system comprises two coil units which are implemented such that the torques produced by the two coil units are minimized.

15. The magnetic resonance scanner as claimed in claim 2, wherein the PET unit includes a plurality of detection units, disposed annularly around the patient bore.

16. The magnetic resonance scanner as claimed in claim 15, wherein the annularly disposed detection units include a recess through which access to the patient bore is defined.

17. The magnetic resonance scanner as claimed in claim 3, wherein the PET unit includes a plurality of detection units, disposed annularly around the patient bore.

18. The magnetic resonance scanner as claimed in claim 17, wherein the annularly disposed detection units include a recess through which access to the patient bore is defined.

19. The magnetic resonance scanner as claimed in claim 4, wherein the PET unit includes a plurality of detection units, disposed annularly around the patient bore.

20. The magnetic resonance scanner as claimed in claim 19, wherein the annularly disposed detection units include a recess through which access to the patient bore is defined.

21. The magnetic resonance scanner as claimed in claim 6, wherein the PET unit includes at least one processing unit for PET data, disposed annularly around the detection units.
